(19) United States
(12) Patent Application Publication
(10) Pub. No.: US 2024/0164373 A1
(43) Pub. Date: May 23, 2024

(54) ANTIMICROBIAL PERFUMING CONSUMER PRODUCTS

(71) Applicant: Firmenich SA, Satigny (CH)

(72) Inventors: Monica BANDERA, Satigny (CH); Yang HUANG, Shanghai (CN)

(21) Appl. No.: 18/551,399

(22) PCT Filed: Mar. 16, 2022
(86) PCT No.: PCT/EP2022/056875

§ 371 Date: Sep. 19, 2023

(30) Foreign Application Priority Data
Mar. 19, 2021 (WO) PCT/CN2021/081823
May 4, 2021 (EP) 21172071.9

(51) Int. Cl. A01N 31/04; A01N 31/02; A01P 1/00

(52) U.S. Cl. CPC A01N 31/04; A01N 31/02; A01P 1/00

(57) ABSTRACT

The present invention is related to perfuming consumer product comprising ingredients selected from the group consisting of: 4-methyl-6-phenyl-2-hexanol, 3-methyl-5-phenyl-1-pentanol and 3,5-dimethyl-5-octen-4-ol for providing an antimicrobial effect. The present invention also relates to methods and further compositions providing the antimicrobial effect.

### TECHNICAL FIELD

The present invention is related to methods and compositions for human or animal body surface and hair cleaning applications, soft and porous substrates like fabric or hard surfaces cleaning applications, deodorant, oral care, or air care applications in order to reduce or eliminate microbes.

### BACKGROUND

Hygiene is a vast subject, which in its fullest meaning goes beyond simple ‘cleanliness’ to include, in addition to products, processes and devices, all circumstances and practices, lifestyle habits and premises that engender and foster a safe and healthy environment. In particular, hygiene refers to conditions and practices that help to maintain health and prevent the spread of diseases and therefore includes a specific set of practices associated with this preservation of health, for example environmental cleaning, sterilization of equipment, hand hygiene, water and sanitation or safe disposal of medical waste.

To improve hygiene, compounds having antimicrobial effects have been developed. However, despite the availability of antimicrobial compounds and compositions, there remains a continuous need to find alternative antimicrobial compositions and active compounds that are suitable for use in such compositions.

### DESCRIPTION

The present disclosure provides a solution to the above mentioned problem by using as an antimicrobial agent, ingredient selected from the group consisting of: 4-methyl-6-phenyl-2-hexanol, 3-methyl-5-phenyl-1-pentanol and 3,5-dimethyl-5-octen-4-ol.

A first aspect of the invention provides the use of a perfuming consumer product comprising ingredients selected from the group consisting of: 4-methyl-6-phenyl-2-hexanol, 3-methyl-5-phenyl-1-pentanol and 3,5-dimethyl-5-octen-4-ol for providing an antimicrobial effect.

A further aspect of the invention provides an antimicrobial composition for use in a perfuming consumer product comprising ingredients selected from the group consisting of: 4-methyl-6-phenyl-2-hexanol, 3-methyl-5-phenyl-1-pentanol and 3,5-dimethyl-5-octen-4-ol.

WO2005004601 refers to certain secondary alcohols being useful as active substances against Gram-positive bacteria. The document does not disclose 4-methyl-6-phenyl-2-hexanol, 4-methyl-6-phenyl-2-hexanol, 3-methyl-5-phenyl-1-pentanol and 3,5-dimethyl-5-octen-4-ol.

WO2006053458 refers to bactericidal effects of certain ingredients on Gram negative bacteria only.

JP2003095816 discloses inhibition zone effects of certain ingredients on S. aureus and E. coli bacteria only.

As shown in the examples below, the perfume ingredients presented herein demonstrate an antimicrobial effect. In some aspects, the antimicrobial effect is bactericidal, wherein bacterial cells are inactivated or killed. Alternatively, in some aspects, the antimicrobial effect is bacteriostatic, wherein the growth rate of bacterial cells is reduced, arrested, or inhibited.

Referring to the examples below, the antimicrobial effect of the perfume ingredients in the antimicrobial compositions was discovered by testing a wide range of concentrations (doses) of individual perfume ingredients, and observing the bactericidal effect against the noted microbial strains (Staphylococcus aureus, Cutibacterium acnes, Corynebacterium tuberculostearicum and Escherichia coli)

The use of the perfuming consumer product or antimicrobial composition as defined herein is particularly advantageous to inactivate microorganisms such as bacteria. The antimicrobial effect is one of the main requirements of hygiene products such as body care or home care products. The antimicrobial effect of the perfuming consumer product or antimicrobial composition provided herein may be determined using any method readily selected by one of ordinary skill in the art. One example of a method for determining the antimicrobial effect of the perfuming consumer product or antimicrobial composition provided herein is the provided in the examples below.

The perfuming consumer product or antimicrobial composition of the invention may comprise 4-methyl-6-phenyl-2-hexanol. By “4-methyl-6-phenyl-2-hexanol” we include any one of its stereoisomers or a mixture thereof.

As mentioned above, WO2005004601 refers to certain secondary alcohols being useful as active substances against Gram-positive bacteria. The document does not disclose 4-methyl-6-phenyl-2-hexanol.

For the sake of clarity, by the expression “any one of its stereoisomers or a mixture thereof”, or the similar, it is meant the normal meaning understood by a person skilled in the art, i.e. that 4-methyl-6-phenyl-2-hexanol can be a pure enantiomer or diastereomer. In other words, 4-methyl-6-phenyl-2-hexanol possesses two stereocenters and each of said stereocenter can have two different stereochemistries (e.g. R or S). 4-methyl-6-phenyl-2-hexanol may even be in the form of a pure enantiomer or in the form of a mixture of enantiomers or diastereoisomers. 4-methyl-6-phenyl-2-hexanol can be in a racemic form or scalemic form. Therefore, 4-methyl-6-phenyl-2-hexanol can be one stereoisomers or in the form of a composition of matter comprising, or consisting of, various stereoisomers.

In particular 4-methyl-6-phenyl-2-hexanol can be (2RS,4SR)-4-methyl-6-phenyl-2-hexanol, (2RS,4RS)-4-methyl-6-phenyl-2-hexanol or mixtures thereof.

The perfuming consumer product or antimicrobial composition of the invention may comprise 3-methyl-5-phenyl-1-pentanol. By “3-methyl-5-phenyl-1-pentanol” we include any one of its stereoisomers or a mixture thereof.

WO2006053458 refers to bactericidal effects of certain ingredients on Gram negative bacteria only.

Where the perfuming consumer product or antimicrobial composition comprises 3-methyl-5-phenyl-1-pentanol, an embodiment of the invention is wherein the antimicrobial effect is against gram positive bacteria. Preferably the gram positive bacteria Cutibacterium acnes or Corynebacterium tuberculostearicum.

For the sake of clarity, by the expression “any one of its stereoisomers or a mixture thereof”, or the similar, it is meant the normal meaning understood by a person skilled in the art, i.e. that 3-methyl-5-phenyl-1-pentanol can be a pure enantiomer. In other words, 3-methyl-5-phenyl-1-pentanol possesses one stereocenter said stereocenter can have two different stereochemistries (e.g. R or S). 3-methyl-5-phenyl-1-pentanol may even be in the form of a pure enantiomer or in the form of a mixture of enantiomers. 3-methyl-5-phenyl-1-pentanol can be in a racemic form or scalemic form. Therefore, 3-methyl-5-phenyl-1-pentanol can be one stereoisomers or in the form of a composition of matter comprising, or consisting of, various stereoisomers.

In particular 3-methyl-5-phenyl-1-pentanol can be (R)-3-methyl-5-phenyl-1-pentanol, (S)-3-methyl-5-phenyl-1-pentanolor mixtures thereof.

The perfuming consumer product or antimicrobial composition of the invention may comprise 3,5-dimethyl-5-octen-4-ol. By “3,5-dimethyl-5-octen-4-ol” we include any one of its stereoisomers or a mixture thereof.

For the sake of clarity, by the expression “any one of its stereoisomers or a mixture thereof”, or the similar, it is meant the normal meaning understood by a person skilled in the art, i.e. that 3,5-dimethyl-5-octen-4-ol can be a pure enantiomer or diastereomer. In other words, 3,5-dimethyl-5-octen-4-ol possesses two stereocenters and each of said stereocenter can have two different stereochemistries (e.g. R or S). 3,5-dimethyl-5-octen-4-ol may even be in the form of a pure enantiomer or in the form of a mixture of enantiomers or diastereoisomers. 3,5-dimethyl-5-octen-4-ol can be in a racemic form or scalemic form. Therefore, 3,5-dimethyl-5-octen-4-ol can be one stereoisomers or in the form of a composition of matter comprising, or consisting of, various stereoisomers.

In particular 3,5-dimethyl-5-octen-4-ol can be (3RS,4RS,5E)-3,5-dimethyl-5-octen-4-ol, (3RS,4SR,5E)-3,5-dimethyl-5-octen-4-ol or mixtures thereof.

In some aspects, the perfuming consumer product or antimicrobial composition provides the antimicrobial effect by inhibiting the growth of bacteria. In one aspect, the antimicrobial effect is an inhibition of growth of a bacterial species selected from the group consisting of: Staphylococcus aureus, Cutibacterium acnes, Corynebacterium tuberculostearicum and Escherichia coli.

In some aspects, the present disclosure provides a use, or a method of use of a composition comprising ingredients selected from the group consisting of: 4-methyl-6-phenyl-2-hexanol, 3-methyl-5-phenyl-1-pentanol and 3,5-dimethyl-5-octen-4-ol. In one embodiment of the invention the ingredient is present in an amount sufficient to provide an antimicrobial effect.

By the term “antimicrobial agent”, it is meant the normal meaning in the art; i.e. an agent which kills microorganism or inhibits their growth.

In some aspects, the present disclosure provides a perfuming consumer product or antimicrobial composition comprising 4-methyl-6-phenyl-2-hexanol and 3-methyl-5-phenyl-1-pentanol.

In some aspects, the present disclosure provides a perfuming consumer product or antimicrobial composition comprising 4-methyl-6-phenyl-2-hexanol and 3,5-dimethyl-5-octen-4-ol.

In some aspects, the present disclosure provides a perfuming consumer product or antimicrobial composition comprising 3-methyl-5-phenyl-1-pentanoland 3,5-dimethyl-5-octen-4-ol.

In some aspects, the present disclosure provides a perfuming consumer product or antimicrobial composition comprising 3-methyl-5-phenyl-1-pentanoland (3RS,4SR,5E)-3,5-dimethyl-5-octen-4-ol.

In some aspects, the present disclosure provides a perfuming consumer product or antimicrobial composition comprising 4-methyl-6-phenyl-2-hexanol, 3-methyl-5-phenyl-1-pentanoland 3,5-dimethyl-5-octen-4-ol.

In an aspect of the invention the perfuming consumer product or antimicrobial composition comprises ingredients each in an amount between 50 ppm and 100,000 ppm.

In some aspects, the perfuming consumer product or antimicrobial composition used in the present disclosure comprises 4-methyl-6-phenyl-2-hexanol in an amount above 50 ppm of the overall composition. Preferably the 4-methyl-6-phenyl-2-hexanol does not exceed 100,000 ppm of the overall composition, preferably 50,000 ppm.

Where the bacterial species is Staphylococcus aureus, preferably the 4-methyl-6-phenyl-2-hexanol is present in an amount above 148 ppm, preferably above 173 ppm.

Where the bacterial species is Cutibacterium acnes, preferably the 4-methyl-6-phenyl-2-hexanol is present in an amount above 99 ppm, preferably above 148 ppm.

Where the bacterial species is Corynebacterium tuberculostearicum, preferably the 4-methyl-6-phenyl-2-hexanol is present in an amount above 900 ppm, preferably above 1300 ppm.

Where the bacterial species is Escherichia coli, preferably the 4-methyl-6-phenyl-2-hexanol is present in an amount above 10000 ppm, preferably above 25,000 ppm.

In some aspects, the perfuming consumer product or antimicrobial composition used in the present disclosure comprises 3-methyl-5-phenyl-1-pentanolin an amount above 50 ppm of the overall composition. Preferably the 3-methyl-5-phenyl-1-pentanoldoes not exceed 100,000 ppm of the overall composition, preferably 50,000 ppm.

Where the bacterial species is Staphylococcus aureus, preferably the 3-methyl-5-phenyl-1-pentanolis present in an amount above 148 ppm, preferably above 185 ppm.

Where the bacterial species is Cutibacterium acnes, preferably the 3-methyl-5-phenyl-1-pentanolis present in an amount above 222 ppm, preferably above 259 ppm.

Where the bacterial species is Corynebacterium tuberculostearicum, preferably the 3-methyl-5-phenyl-1-pentanolis present in an amount above 148 ppm, preferably above 200 ppm.

Where the bacterial species is Escherichia coli, preferably the 3-methyl-5-phenyl-1-pentanolis present in an amount above 333 ppm, preferably above 360 ppm.

In some aspects, the perfuming consumer product or antimicrobial composition used in the present disclosure comprises 3,5-dimethyl-5-octen-4-ol in an amount above 50 ppm of the overall composition. Preferably the 3,5-dimethyl-5-octen-4-ol does not exceed 100,000 ppm of the overall composition, preferably 50,000 ppm.

Where the bacterial species is Staphylococcus aureus, preferably the 3,5-dimethyl-5-octen-4-ol is present in an amount above 1300 ppm, preferably above 1533 ppm.

Where the bacterial species is Cutibacterium acnes, preferably the 3,5-dimethyl-5-octen-4-ol is present in an amount above 1300 ppm, preferably above 1767 ppm.

Where the bacterial species is Corynebacterium tuberculostearicum, preferably the 3,5-dimethyl-5-octen-4-ol is present in an amount above 2000 ppm, preferably above 2667 ppm.

Where the bacterial species is Escherichia coli, preferably the 3,5-dimethyl-5-octen-4-ol is present in an amount above 3000 ppm, preferably above 3500 ppm.

The use of the perfuming consumer product or antimicrobial composition as defined herein is particularly advantageous to limit or control the growth of microorganism such as bacteria. The antimicrobial effect is one of the main requirements of hygiene products such as body care or home care products.

As mentioned above, the disclosure concerns the use of the perfuming consumer product or antimicrobial composition as defined above as an antimicrobial agent.

A further aspect of the invention provides the perfuming consumer product or antimicrobial composition comprising one or more ingredients selected from the group consisting of: 4-methyl-6-phenyl-2-hexanol, 3-methyl-5-phenyl-1-pentanoland 3,5-dimethyl-5-octen-4-ol. as an antimicrobial composition. An embodiment of this invention is wherein the perfuming consumer product or antimicrobial composition comprises all combinations of the ingredients from the group as outlined in the aspects of the invention as defined herein. An additional aspect of the invention is wherein the substrate is treated with the composition in an amount sufficient to provide an antimicrobial effect.

In some aspects, the present disclosure provides a non-therapeutic method of affecting microbial activity, the method comprising treating a substrate comprising microbes with a perfuming consumer product or antimicrobial composition comprising ingredients selected from the group consisting of 4-methyl-6-phenyl-2-hexanol, 3-methyl-5-phenyl-1-pentanoland 3,5-dimethyl-5-octen-4-ol. An embodiment of this invention is wherein the perfuming consumer product or antimicrobial composition comprises all combinations of the ingredients from the group as outlined in the aspects of the invention as defined herein. An additional aspect of the invention is wherein the substrate is treated with the perfuming consumer product or antimicrobial composition in an amount sufficient to provide an antimicrobial effect.

By the term “affecting”, it is meant the inhibition of growth of microorganisms or the killing of microorganisms.

In some aspects, the present disclosure provides a perfuming composition comprising:
a composition comprising 4-methyl-6-phenyl-2-hexanol and a least one compound selected from the group consisting of 3-methyl-5-phenyl-1-pentanoland 3,5-dimethyl-5-octen-4-ol, wherein the composition is present in an amount sufficient to provide an antimicrobial effect;
at least one ingredient selected from the group consisting of a perfumery carrier, a perfuming co-ingredient and mixtures thereof; and
optionally at least one perfumery adjuvant.

In some aspects, the present disclosure provides a perfuming composition comprising:
a composition comprising 3-methyl-5-phenyl-1-pentanoland a least one compound selected from the group consisting of 4-methyl-6-phenyl-2-hexanol and 3,5-dimethyl-5-octen-4-ol, wherein the composition is present in an amount sufficient to provide an antimicrobial effect;
at least one ingredient selected from the group consisting of a perfumery carrier, a perfuming co-ingredient and mixtures thereof; and
optionally at least one perfumery adjuvant.

In some aspects, the present disclosure provides a perfuming composition comprising:
a composition comprising 3,5-dimethyl-5-octen-4-ol and a least one compound selected from the group consisting of 4-methyl-6-phenyl-2-hexanol and 3-methyl-5-phenyl-1-pentanol, wherein the composition is present in an amount sufficient to provide an antimicrobial effect;
at least one ingredient selected from the group consisting of a perfumery carrier, a perfuming co-ingredient and mixtures thereof; and
optionally at least one perfumery adjuvant.

In some aspects, the present disclosure provides a perfuming composition comprising:
a composition comprising 4-methyl-6-phenyl-2-hexanol and 3-methyl-5-phenyl-1-pentanol, wherein the composition is present in an amount sufficient to provide an antimicrobial effect;
at least one ingredient selected from the group consisting of a perfumery carrier, a perfuming co-ingredient and mixtures thereof; and
optionally at least one perfumery adjuvant.

In some aspects, the present disclosure provides a perfuming composition comprising:
a composition comprising 4-methyl-6-phenyl-2-hexanol and 3,5-dimethyl-5-octen-4-ol, wherein the composition is present in an amount sufficient to provide an antimicrobial effect;
at least one ingredient selected from the group consisting of a perfumery carrier, a perfuming co-ingredient and mixtures thereof; and
optionally at least one perfumery adjuvant.

In some aspects, the present disclosure provides a perfuming composition comprising:
a composition comprising 3-methyl-5-phenyl-1-pentanoland 3,5-dimethyl-5-octen-4-ol, wherein the composition is present in an amount sufficient to provide an antimicrobial effect;
at least one ingredient selected from the group consisting of a perfumery carrier, a perfuming co-ingredient and mixtures thereof; and
optionally at least one perfumery adjuvant.

An embodiment of the perfuming composition of the invention is wherein comprises all combinations of the ingredients from the group as outlined in the aspects of the invention as defined herein.

An embodiment of the perfuming composition of the invention is wherein composition is present in an amount sufficient to provide an antimicrobial effect.

In some embodiments, the amount sufficient to provide an antimicrobial effect has no impact on the overall odor profile of the composition.

By “perfumery carrier” we mean here a material which is practically neutral from a perfumery point of view, i.e. which does not significantly alter the organoleptic properties of perfuming ingredients. The carrier may be a liquid or a solid.

As liquid carrier one may cite, as non-limiting examples, an emulsifying system, i.e. a solvent and a surfactant system, or a solvent commonly used in perfumery. A detailed description of the nature and type of solvents commonly used in perfumery cannot be exhaustive. However, one can cite as non-limiting examples solvents such as butylene or propylene glycols, glycerol, dipropyleneglycol and its monoether, 1,2,3-propanetriyl triacetate, dimethyl glutarate, dimethyl adipate 1,3-diacetyloxypropan-2-yl acetate, diethyl phthalate, isopropyl myristate, benzyl benzoate, benzyl alcohol, 2-(2-ethoxyethoxy)-1-ethano, tri-ethyl citrate or mixtures thereof, which are the most commonly used. For the compositions which comprise both a perfumery carrier and a perfumery co-ingredient, other suitable perfumery carriers than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company), or hydrogenated castors oils such as those known under the trademark Cremophor® RH 40 (origin: BASF).

As solid carrier it is meant a material where the perfuming composition or some element of the perfuming composition can be chemically or physically bound. In general such solid carrier are employed either to stabilize the composition, either to control the rate of evaporation of the compositions or of some ingredients. The employment of solid carrier is of current use in the art and a person skilled in the art knows how to reach the desired effect. However by way of non-limiting example as solid carriers one may cite absorbing gums or polymers or inorganic material, such as porous polymers, cyclodextrines, wood based materials, organic or inorganic gels, clays, gypsum talc or zeolites.

As other non-limiting example of solid carrier one may cite encapsulating materials. Examples of such materials may comprise wall-forming and plasticizing materials, such as mono, di- or trisaccharides, natural or modified starches, hydrocolloids, cellulose derivatives, polyvinyl acetates, polyvinylalcohols, proteins or pectins, or yet the materials cited in reference texts such as H. Scherz, Hydrokolloide: Stabilisatoren, Dickungs-und Geliermittel in Lebensmitteln, Band 2 der Schriftenreihe Lebensmittelchemie, Lebensmittelqualität, Behr's Verlag Gmbh & Co., Hamburg, 1996. The encapsulation is a well-known process to a person skilled in the art, and may be performed, for instance, using techniques such as spray-drying, agglomeration or yet extrusion; or consists of a coating encapsulation, including coacervation and complex coacervation technique. As non-limiting examples one may cite in particular the core-shell encapsulation with resins of the aminoplast, polyamide, polyester, polyurea or polyurethane type or a mixture thereof (all of the resins are well known to a person skilled in the art) using techniques like phase separation process induced by polymerization, by interfacial polymerization, by coacervation or altogether (all of the techniques are have been described in the prior art), and optionally in presence of polymeric stabilizer or a cationic copolymer.

In particular, as resins one may cite the ones produced by the polycondensation of an aldehyde (e.g. formaldehyde, 2,2-dimethoxyethanal, glyoxal, glyoxylic acid or glycolaldehyde and mixtures thereof) with an amine, namely urea, benzoguanamine, glycoluryl, melamine, methylol melamine, methylated methylol melamine, guanazole and the like, as well as mixtures thereof. Alternatively one may use preformed resins alkylolated polyamines such as those commercially available under the trademark Urac® (origin: Cytec Technology Corp.), Cymel® (origin: Cytec Technology Corp.), Urecoll® or Luracoll® (origin: BASF).

In particular, as resins one may cite the ones produced by the polycondensation of an a polyol, like glycerol, and a polyisocyanate, like a trimer of hexamethylene diisocyanate, a trimer of isophorone diisocyanate or xylylene diisocyanate or a Biuret of hexamethylene diisocyanate or a trimer of xylylene diisocyanate with trimethylolpropane (known with the tradename of Takenate®, origin: Mitsui Chemicals), among which a trimer of xylylene diisocyanate with trimethylolpropane and a Biuret of hexamethylene diisocyanate.

Some of the seminal literature related to the encapsulation of perfumes by polycondensation of amino resins, namely melamine based resins, with aldehydes is represented by articles such as those published by K. Dietrich et al. in Acta Polymerica, 1989, vol. 40, pages 243, 325 and 683, as well as 1990, vol. 41, page 91. Such articles already describe the various parameters affecting the preparation of such core-shell microcapsules following prior art methods that are also further detailed and exemplified in the patent literature. US 4′396′670, to the Wiggins Teape Group Limited is a pertinent early example of the latter. Since then, many other authors and creators have enriched the literature in this field and it would be impossible to cover all published developments here, but the general knowledge in this type of encapsulation is very significant. More recent publications of pertinence, which also address the suitable uses of such microcapsules, are represented for example by the article of H. Y. Lee et al. in Journal of Microencapsulation, 2002, vol. 19, pages 559-569, international patent publication WO 01/41915 or yet the article of S. Bône et al. in Chimia, 2011, vol. 65, pages 177-181. Perfuming co-ingredients, when present in the perfuming composition, are other than 4-methyl-6-phenyl-2-hexanol, 3-methyl-5-phenyl-1-pentanoland 3,5-dimethyl-5-octen-4-ol.

Moreover, by “perfuming co-ingredient” it is meant here a compound, which is used in a perfuming preparation or a composition to impart a hedonic effect. In other words such a co-ingredient, to be considered as being a perfuming one, must be recognized by a person skilled in the art as being able to impart or modify in a positive or pleasant way the odor of a composition, and not just as having an odor.

The nature and type of the perfuming co-ingredients do not warrant a more detailed description here, which in any case would not be exhaustive, the skilled person being able to select them on the basis of his general knowledge and according to intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulphurous heterocyclic compounds and essential oils, and the perfuming co-ingredients can be of natural or synthetic origin.

In particular one may cite perfuming co-ingredients which are commonly used in perfume formulations, such as:
Aldehydic ingredients: decanal, dodecanal, 2-methyl-undecanal, 10-undecenal, octanal and/or nonenal;
Aromatic-herbal ingredients: eucalyptus oil, camphor, eucalyptol, menthol and/or alpha-pinene;
Balsamic ingredients: coumarine, ethylvanillin and/or vanillin;
Citrus ingredients: dihydromyrcenol, citral, orange oil, linalyl acetate, citronellyl nitrile, orange terpenes, limonene, 1-P-menthen-8-yl acetate and/or 1,4(8)-P-menthadiene;
Floral ingredients: Methyl dihydrojasmonate, linalool, Citronellol, phenylethanol, 3-(4-tert-butylphenyl)-2-methylpropanal, hexylcinnamic aldehyde, benzyl acetate, benzyl salicylate, tetrahydro-2-isobutyl-4-methyl-4(2H)-pyranol, beta ionone, methyl 2-(methylamino)benzoate, (E)-3-methyl-4-(2,6,6-trimethyl-2-cyclohexen-1-yl)-3-buten-2-one, hexyl salicylate, 3,7-dimethyl-1,6-nonadien-3-ol, 3-(4-isopropylphenyl)-2-methylpropanal, verdyl acetate, geraniol, P-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate, high cis methyl dihydrojasmonate, 3-methyl-5-phenyl-1-pentanol, verdyl proprionate, geranyl acetate, tetrahydro linalool, cis-7-P-menthanol, Propyl (S)-2-(1,1-dimethylpropoxy)propanoate, 2-methoxynaphthalene, 2,2,2-trichloro-1-phenylethyl acetate, 4/3-(4-hydroxy-4-methylpentyl)-3-cyclohexene-1-carbaldehyde, amylcinnamic aldehyde, 4-phenyl-2-butanone, isononyle acetate, 4-(1,1-diméthyléthyl)-1-cyclohexyl acetate, verdyl isobutyrate and/or mixture of methylionones isomers;
Fruity ingredients: gamma ungamma-dodecalactoneactone, 4-decanolide, ethyl 2-methyl-pentanoate, hexyl acetate, ethyl 2-methylbutanoate, gamma nonalactone, allyl heptanoate, 2-phenoxyethyl isobutyrate, ethyl 2-methyl-1,3-dioxolane-2-acetate and/or diethyl 1,4-cyclohexane dicarboxylate;
Green ingredients: 2,4-Dimethyl-3-cyclohexene-1-carbaldehyde, 2-tert-butyl-1-cyclohexyl acetate, styrallyl acetate, allyl (2-methylbutoxy)acetate, 4-methyl-3-decen-5-ol, diphenyl ether, (Z)-3-hexen-1-ol and/or 1-(5,5-dimethyl-1-cyclohexen-1-yl)-4-penten-1-one;
Musk ingredients: 1,4-dioxa-5,17-cycloheptadecanedione, pentadecenolide, 3-Methyl-5-cyclopentadecen-1-one, 1,3,4,6,7,8-hexahydro-4,6,6,7,8,8-hexamethyl-cyclopenta-g-2-benzopyrane, (1S, 1′R)-2-[1-(3′,3′-dimethyl-1′-cyclohexyl)ethoxy]-2-methylpropyl propanoate, pentadecanolide and/or (1S, 1′R)-[1-(3′,3′-Dimethyl-1′-cyclohexyl)ethoxycarbonyl]methyl propanoate;
Woody ingredients: 1-(octahydro-2,3,8,8-tetramethyl-2-naphtalenyl)-1-ethanone, patchouli oil, terpenes fractions of patchouli oil, (1′R,E)-2-ethyl-4-(2′,2′,3′-trimethyl-3′-cyclopenten-1′-yl)-2-buten-1-ol, 2-ethyl-4-(2,2,3-trimethyl-3-cyclopenten-1-yl)-2-buten-1-ol, Methyl cedryl ketone, 5-(2,2,3-trimethyl-3-cyclopentenyl)-3-methylpentan-2-ol, 1-(2,3,8,8-tetramethyl-1,2,3,4,6,7,8,8a-octahydronaphthalen-2-yl)ethan-1-one and/or isobornyl acetate;
Other ingredients (e.g. amber, powdery spicy or watery): dodecahydro-3a,6,6,9a-tetramethyl-naphtho[2,1-b]furan and any of its stereoisomers, heliotropin, anisic aldehyde, eugenol, cinnamic aldehyde, clove oil, 3-(1,3-benzodioxol-5-yl)-2-methylpropanal and/or 3-(3-isopropyl-1-phenyl)butanal.

Perfuming co-ingredients may not be limited to the above mentioned, and many other of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that the co-ingredients may also be compounds known to release in a controlled manner various types of perfuming compounds.

By “perfumery adjuvant” what is meant is an ingredient capable of imparting additional added benefit such as a color, a particular light resistance, chemical stability, etc. A detailed description of the nature and type of adjuvant commonly used in perfumery cannot be exhaustive, but it has to be mentioned that the ingredients are well known to a person skilled in the art. However, one may cite as specific non-limiting examples the following: viscosity agents (e.g. surfactants, thickeners, gelling and/or rheology modifiers), stabilizing agents (e.g. preservatives, antioxidant, heat/light and or buffers or chelating agents, such as BHT), color agents (e.g. dyes and/or pigments), preservative (e.g. antibacterial or antimicrobial or antifungi or anti irritant agents), abrasives, skin cooling agents, fixatives, insect repellants, ointments, vitamins and mixture thereof.

It is understood that a person skilled in the art is perfectly able to design optimal formulations for the desired effect by admixing the above mentioned components of a perfuming composition, simply by applying the standard knowledge of the art as well as by trial and error methodologies.

A perfuming composition consisting of at least one antimicrobial composition as defined above and at least one perfumery carrier represents a particular aspect of the disclosure as well as a perfuming composition comprising at least one antimicrobial composition as defined above, at least one perfumery carrier, at least one perfumery co-ingredient, and optionally at least one perfumery adjuvant.

For the sake of clarity, it is also understood that any mixture resulting directly from a chemical synthesis, e.g. a reaction medium without an adequate purification, in which the compound of the present disclosure would be involved as a starting, intermediate or end-product could not be considered as a perfuming composition according to the disclosure as far as the mixture does not provide the inventive compound in a suitable form for perfumery. Thus, unpurified reaction mixtures are generally excluded from the present disclosure unless otherwise specified.

Furthermore, the antimicrobial composition, as defined above, can also be advantageously used in all the fields of modern perfumery, i.e. fine or functional perfumery, to positively impart or modify the odor of a consumer product into which the compound (I) is added.

The above-composition can be added as such or as part of the present disclosure's perfuming composition.

For the sake of clarity, it has to be mentioned that, by “perfuming consumer product” it is meant a consumer product which is expected to deliver at least a pleasant perfuming effect to the surface to which it is applied (e.g. skin, hair, textile, or home surface) or in the air. In other words, a perfuming consumer product according to the disclosure is a perfumed consumer product which comprises the functional formulation, as well as optionally additional benefit agents, corresponding to the desired consumer product, e.g. a detergent or an air freshener, and an olfactive effective amount of at least one disclosure's compound. For the sake of clarity, the perfuming consumer product is a non-edible product.

The nature and type of the constituents of the perfuming consumer product do not warrant a more detailed description here, which in any case would not be exhaustive, the skilled person being able to select them on the basis of his general knowledge and according to the nature and the desired effect of the product.

Non-limiting examples of suitable perfuming consumer product can be a perfume, such as a fine perfume, a splash or eau de perfume, a cologne or a shave or after-shave lotion; a fabric care product, such as a liquid or solid detergent, a fabric softener, a fabric refresher, an ironing water, a paper, or a bleach, carpet cleaners, curtain-care products; a body-care product, such as a hair care product (e.g. a shampoo, a coloring preparation or a hair spray, a color care product, hair shaping product, a dental care product), a disinfectant, an intimate care product; a cosmetic preparation (e.g. a skin cream or lotion, a vanishing cream or a deodorant or antiperspirant (e.g. a spray or roll on), hair remover, tanning or sun or after sun product, nail products, skin cleansing, a makeup); or a skin-care product (e.g. a perfumed soap, shower or bath mousse, oil or gel, or a hygiene product or a foot/hand care products); an air care product, such as an air freshener or a “ready to use” powdered air freshener which can be used in the home space (rooms, refrigerators, cupboards, shoes or car) and/or in a public space (halls, hotels, malls, etc.); or a home care product, such as a mold remover, furnisher care, wipe, a dish detergent or hard-surface (e.g. a floor, bath, sanitary or a windows) detergent; a leather care product; a car care product, such as a polish, waxes or a plastic cleaners. Alternatively, in some aspects, the consumer products are body care or home care products.

Some of the above-mentioned perfuming consumer products may represent an aggressive medium for the disclosure's composition, so that it may be necessary to protect the latter from premature decomposition, for example by encapsulation or by chemically binding it to another chemical which is suitable to release the disclosure's ingredient upon a suitable external stimulus, such as an enzyme, light, heat or a change of pH.

The proportions in which the composition according to the disclosure can be incorporated into the various aforementioned products or compositions vary within a wide range of values. These values are dependent on the nature of the article to be perfumed and on the desired organoleptic effect as well as on the nature of the co-ingredients when the compounds according to the disclosure are mixed with perfuming co-ingredients, solvents or additives commonly used in the art.

For example, in the case of perfuming compositions, typical concentrations are in the order of 0.001% to 10% by weight, or even more, of the disclosure's composition based on the weight of the composition into which they are incorporated. Concentrations lower than these, such as in the order of 0.01% to 1% by weight, can be used when these compounds are incorporated into perfuming consumer products, percentage being relative to the weight of the article.

In some aspects, the present disclosure provides the use or a method of using a composition comprising 4-methyl-6-phenyl-2-hexanol, 3-methyl-5-phenyl-1-pentanoland 3,5-dimethyl-5-octen-4-ol for the preparation of an antimicrobially active consumer product.

By the expression “antimicrobially active consumer product” it is meant the normal meaning in the art: i.e. a consumer product which is expected to inhibit the growth of microorganism or to kill the microorganism present on the surface to which it is applied (e.g. skin, hair, textile, or home surface).

The disclosure also provides particular methods of use including counteracting microbial activity in a perfuming or consumer product in particular by circumventing the presence of chlorinated biocides in such products.

The invention will now be described in further detail by way of the following examples which illustrate the benefits and advantages of the present invention.

### EXAMPLES

### Example 1: Antimicrobial Activity of the Ingredients

Method

Preparation of Bacterial Suspensions

Bacterial suspensions of Escherichia coli and Staphylococcus aureus were prepared as follows. Stock cultures stored at −80° C. were subcultured onto agar plate media, and incubated at 37ºC for 24 h to obtain single colonies. Single colonies of the primary cultures were inoculated into broth media and incubated at 37ºC, 160 rpm overnight. Aliquots of overnight cultures were inoculated into 50 ml of fresh broth media, and incubated at 37° C., 160 rpm. When the OD600 nm reached the target value for each strain (see Table 1), cells were harvested by centrifugation at 5000 rpm for 10 min, and then resuspended in the same fresh broth media at the same volume before the centrifugation. Aliquots (1.1 ml) of each cell suspension were diluted in 200 ml of the same broth media as the bacterial solutions for the MIC test.

TABLE 1
Media, aliquots of overnight culture, and target OD of broth cultures for
the preparation of bacterial suspensions of E. coli and S. aureus

| | | | Aliquots of | |
|---|---|---|---|---|
| | Agar plate | Broth | Overnight | |
| Strains | media | Media | Culture | Target OD |
| Escherichia coli | TSA^{a} | MH^{b} | 0.5 ml | 0.3 |
| ATCC 25922 | | | | |
| Escherichia coli | TSA^{a} | MH^{b} | 0.5 ml | 0.3 |
| DSM 787 | | | | |
| Staphylococcus aureus | TSA | MH | 0.5 ml | 0.4 |
| ATCC 6538 | | | | |

Note:
^{a}TSA, Tryptic Soy Agar (BD Cat No. 236950),
^{b}MH, Mueller Hinton Broth (BD Cat No. 211443)

Bacterial suspension of the Corynebacterium tuberculostearicum C1 strain was prepared as follows: Stock culture (stored at −80° C.) was subcultured onto tryptic soy agar plates with 0.5% Tween 80 (TSA-TW80), and subsequently incubated at 37ºC for 48 h. The primary cultures were subcultured again onto TSA-TW80 to prepare the secondary cultures. One single colony of the secondary culture was then selected and used to inoculate 50 ml brain heart infusion broth with 0.5% Tween 80 (BHI-TW80). The inoculated broth was incubated at 37° C. and 180 rpm overnight. Aliquots (2 mL) of the overnight culture were inoculated into 50 ml of fresh BHI-TW80 broth media, and incubated at 37° C., 160 rpm. When the OD600 nm reached the target value of 4.0, cells were harvested by centrifugation at 5000 rpm for 10 min, and then resuspended in the fresh BHI-TW80 broth media at the same volume before the centrifugation. Aliquots (1 ml) of each cell suspension were diluted in 200 ml of fresh BHI-TW80 broth media as the bacterial solutions for the MIC test.

Bacterial solutions of anaerobic Cutibacterium acnes DSM 1897 were prepared for MIC test as follows. Stock culture stored at −80° C. were subcultured onto Columbia blood agar (CBA, Biomerieux) plates, and incubated at 37° C. anaerobically for 4 d to obtain single colonies. One loop (1 uL) of the 4 d culture to 9.5 mL Wilkins-Chalgren broth (WC, OXOID) and incubated at 37° C., anerobically for 24 h. Aliquots (2 mL) of the 24 h cultures were inoculated into 100 ml of fresh WC broth media, and incubated at 37ºC, anaerobically for 18 h. Aliquots (1.4 ml) of the overnight culture were diluted in 100 ml of WC broth media as the bacterial solutions for the MIC test.

Preparation of Sample Solutions

(2E)-2-hexenal. was used as the reference material for MIC test of aerobic strains, whereas carvacrol was used for anaerobic strains.

Sample solutions of test materials and reference material were prepared in ethanol for MIC test for E. coli and S. aureus strains or in DMSO for C. tuberculostearicum and C. acnes strains. In brief, stock solutions of 1% and 20% were prepared in each solvent, and then seven serial dilutions of each stock solution were prepared in each solvent to obtain a total of 16 solutions of the test material or the reference material. Aliquot (10 μl) of each solution was used for MIC test. The tested final concentrations of each material were 29, 44, 66, 99, 148, 222, 333, 500, 590, 900, 1300, 2000, 3000, 4500, 6700, 10000 ppm.

MIC Test Procedures

MIC test were performed in 96 well plates. Table 2 shows the schematic positions of sample solutions in 96 well plates. Column 1 contained bacterial solution only (the positive growth control), and column 12 contained growth media only (the negative growth control). Aliquots (10 μl) of sample solutions were mixed with 190 μl of bacterial solutions in growth media, at concentrations of 10⁵ to 10⁶ cfu/ml, in wells of the 96 well plates. Three replicates for each solution.

The 96 wells plates were incubated at 37° C., 160 rpm overnight. After incubation, wells of 96 well plates were examined. Turbid wells were regarded as an indication of microbial growth.

Minimal inhibitory concentration (MIC) was determined as the lowest concentration where no growth was observed. Average MIC value of the three replicates was calculated against each strain.

The results are shown in the tables below:

TABLE 1
Schematic positions of samples in 96 well plates

| | | | | | | | | | | |
|---|---|---|---|---|---|---|---|---|---|---|
| A | Positive | 590 | ppm | 29 | ppm | 590 | ppm | 29 | ppm | Negative |
| B | growth | 900 | ppm | 44 | ppm | 900 | ppm | 44 | ppm | growth |
| C | control | 1300 | ppm | 66 | ppm | 1300 | ppm | 66 | ppm | control |
| D | | 2000 | ppm | 99 | ppm | 2000 | ppm | 99 | ppm | |
| E | | 3000 | ppm | 148 | ppm | 3000 | ppm | 148 | ppm | |
| F | | 4500 | ppm | 222 | ppm | 4500 | ppm | 222 | ppm | |
| G | | 6700 | ppm | 333 | ppm | 6700 | ppm | 333 | ppm | |
| H | | 10000 | ppm | 500 | ppm | 10000 | ppm | 500 | ppm | |

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | |
|---|---|
| Test material | Reference material |

TABLE 2

| | Minimal inhibitory concentration (ppm) | | | | |
|---|---|---|---|---|---|
| | Staphylococcus | Cutibacterium | Corynebacterium | Escherichia | Escherichia |
| Test | aureus | acnes | tuberculostearicum | coli | coli |
| material | ATCC 6538 | DSM 1897 | C1 | ATCC 25922 | DSM 787 |
| 4-methyl- | 173 | 148 | 1300 | >10000 | |
| 6-phenyl- | | | | | |
| 2-hexanol | | | | | |
| 3-methyl- | 185 | 259 | 200 | | 360 |
| 5-phenyl- | | | | | |
| 1-pentanol | | | | | |
| 3,5-dimethyl- | 1533 | 1767 | 2667 | 3500 | |
| 5-octen- | | | | | |
| 4-ol | | | | | |

1. A method for providing an antimicrobial effect to a surface, the method comprising applying a perfuming consumer product comprising one or more ingredients selected from the group consisting of: 4-methyl-6-phenyl-2-hexanol, 3-methyl-5-phenyl-1-pentanol and 3,5-dimethyl-5-octen-4-ol to the surface.

2. The method according to claim 1 wherein the antimicrobial effect is an inhibition of growth of a bacterial species selected from the group consisting of: Staphylococcus aureus, Cutibacterium acnes, Corynebacterium tuberculostearicum and Escherichia coli.

3. The method according to claim 1, wherein the perfuming consumer product comprises one or more of the ingredients each in an amount between 50 ppm and 100,000 ppm.

4. The method according to claim 1, wherein the perfuming consumer product further comprises at least one ingredient selected from the group consisting of a perfumery carrier, a perfuming co-ingredient and mixtures thereof; and optionally at least one perfumery adjuvant.

5. The method according to claim 1, wherein the perfuming consumer product is a perfume, a fabric care product, a body-care product, a cosmetic preparation, a skin-care product, an air care product or a home care product.

6. The method according to claim 1, wherein the perfuming consumer product comprises 4-methyl-6-phenyl-2-hexanol and at least one compound selected from the group consisting of 3-methyl-5-phenyl-1-pentanoland 3,5-dimethyl-5-octen-4-ol.

7. The method according to claim 1, wherein the perfuming consumer product comprises 3-methyl-5-phenyl-1-pentanol and at least one compound selected from the group consisting of 4-methyl-6-phenyl-2-hexanol, and 3,5-dimethyl-5-octen-4-ol.

8. The method according to claim 1, wherein the perfuming consumer product comprises 3,5-dimethyl-5-octen-4-ol and at least one compound selected from the group consisting of 4-methyl-6-phenyl-2-hexanol and 3-methyl-5-phenyl-1-pentanol.

9. The method according to claim 1, wherein the perfuming consumer product comprises 4-methyl-6-phenyl-2-hexanol and 3-methyl-5-phenyl-1-pentanol.

10. The method according to claim 1, wherein the perfuming consumer product comprises 4-methyl-6-phenyl-2-hexanol and 3,5-dimethyl-5-octen-4-ol.

11. The method according to claim 1, wherein the perfuming consumer product comprises 3-methyl-5-phenyl-1-pentanoland 3,5-dimethyl-5-octen-4-ol.

12. The method according to claim 1, wherein the perfuming consumer product comprises 4-methyl-6-phenyl-2-hexanol, 3-methyl-5-phenyl-1-pentanoland 3,5-dimethyl-5-octen-4-ol.

13. A perfuming composition comprising:
a composition comprising 4-methyl-6-phenyl-2-hexanol and a least one compound selected from the group consisting of 3-methyl-5-phenyl-1-pentanoland 3,5-dimethyl-5-octen-4-ol, wherein the composition is present in an amount sufficient to provide an antimicrobial effect;
at least one ingredient selected from the group consisting of a perfumery carrier, a perfuming co-ingredient and mixtures thereof; and
optionally at least one perfumery adjuvant.

14. A perfuming composition comprising:
a composition comprising 3-methyl-5-phenyl-1-pentanol and a least one compound selected from the group consisting of 4-methyl-6-phenyl-2-hexanol and 3,5-dimethyl-5-octen-4-ol, wherein the composition is present in an amount sufficient to provide an antimicrobial effect;
at least one ingredient selected from the group consisting of a perfumery carrier, a perfuming co-ingredient and mixtures thereof; and
optionally at least one perfumery adjuvant.

15. A perfuming composition comprising:
a composition comprising 3,5-dimethyl-5-octen-4-ol and a least one compound selected from the group consisting of 4-methyl-6-phenyl-2-hexanol and 3-methyl-5-phenyl-1-pentanol, wherein the composition is present in an amount sufficient to provide an antimicrobial effect;
at least one ingredient selected from the group consisting of a perfumery carrier, a perfuming co-ingredient and mixtures thereof; and
optionally at least one perfumery adjuvant.

16. The composition according to claim 13,-wherein the 4-methyl-6-phenyl-2-hexanol is present in an amount between 50 ppm and 100,000 ppm.

17. The composition according to claim 14,-wherein the 3-methyl-5-phenyl-1-pentanol is present in an amount between 50 ppm and 100,000 ppm.

18. The composition according to claim 15,-wherein the 3,5-dimethyl-5-octen-4-ol is present in an amount between 50 ppm and 100,000 ppm.
